(19) United States
(12) Patent Application Publication
(10) Pub. No.: US 2007/0265519 A1
(43) Pub. Date: Nov. 15, 2007

(54) METHOD AND MEDICAL IMAGING SYSTEM FOR GUIDING A USER THROUGH A DATA ACQUISITION PROCEDURE

(76) Inventor: Gerhard Bittner, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/740,316

(22) Filed: Apr. 26, 2007

(30) Foreign Application Priority Data
Apr. 26, 2006 (DE) 10 2006 019 454.3

(51) Int. Cl. A61B 5/05

(52) U.S. Cl. 600/407

(57) ABSTRACT

In a method and medical imaging system for surgical/interventional guidance during the operation of a medical imaging system, in particular a magnetic resonance apparatus, at least one item of information regarding operation of the medical imaging system is shown to a user, the shown information depending on an internal operating state of the medical imaging system.

### BACKGROUND OF THE INVENTION

1. Field of the Invention

The present invention concerns a method for guiding a user through a data acquisition procedure involving a medical imaging system, in particular guidance through an examination with a magnetic resonance apparatus, as well as a medical imaging system, in particular a magnetic resonance apparatus, operating according to the method.

2. Description of the Prior Art

Many medical imaging systems are complex apparatuses for which numerous details must be observed during the operation in order to ensure a correct functioning of the imaging systems and the safety for user and patient. In particular in the case of magnetic resonance apparatuses, the operation must be conducted with particular care and attention since operator errors can lead to considerable harm for patient and/or apparatus due to the strong static magnetic field and the considerable power of the radio-frequency magnetic fields that are radiated during acquisition of MR data from the patient.

So that the necessary safety is ensured, a user must be trained in a complicated manner, for example by study in the very comprehensive usage instructions or through special introductions regarding the operation of the medical imaging systems. A series of warning signs that indicate the most important danger situations is additionally often mounted near the imaging systems.

In spite of elaborate usage instructions and reasonably applied warning instructions, a user is sometimes overextended, for example because the user does not sufficiently recall previous teachings, or overlooks the always-identical warning instructions, which can lead to operator errors.

### SUMMARY OF THE INVENTION

An object of the present invention is to provide a method for guiding a user through a data acquisition procedure (examination) involving a medical imaging system, with which a user is aided (supported) in an effective manner in the correct operation of the medical imaging system. Furthermore, an object of the invention is to provide medical imaging system with which a user is supported in an effective manner in the procedure.

The object is inventively achieved by a method for guidance through (during) the operation of a medical imaging system, in particular a magnetic resonance apparatus, wherein at least one item of information for operation of the magnetic resonance apparatus is made perceptible to a user, whereby the presented information depends on an internal operating state of the medical imaging system.

In this manner a user can, in a targeted manner, be shown information that is adapted to the internal operating state of the medical imaging system. Much information for operation guidance does not have to be observed during the entire operation, but only has to be noted selectively at specific points in time, i.e. given specific operating states of the medical imaging system. For example, with a magnetic resonance apparatus, an operating instruction that describes precaution measures for the positioning a patient on the patient bed of the magnetic resonance apparatus must only be taken into account in the preparation of the patient in the measurement chamber before the start of the actual measurement and again after implementation of the measurement. With the inventive method the information can now be superimposed in a targeted manner depending on the operating state of the medical imaging system, for example at the points in time at which the information is relevant.

A sequence of items of information regarding operation of the medical imaging system is preferably presented to the user, the respective presented information depending on the internal operating state of the medical imaging system. In this manner a user is conducted in the surgical/interventional guidance since the respective information steps important for operation of the medical imaging system can be presented sequentially. The information of the sequence of information is determined from the current internal operating state of the medical imaging system.

The at least one item of information that is presented is output acoustically and/or optically in various embodiments. The presentation form of the information, for example, can be adapted to the spatial conditions and the desire of the user.

The at least one item of information that is presented is preferably shown in a measurement chamber in which the medical imaging system is located. In this manner a user can be directed in the measurement compartment in which he typically must implement many work steps before, during and after a measurement and therefore must attend to many details in the operation. The user can be effectively directed in this manner with the sequential presentation of a sequence of information for operation.

The at least one item of information is optically presented on a wall of the measurement chamber in a preferred embodiment of the method. For example, the information can be projected from a projector on the wall of the measurement chamber or can be shown on a display screen that is mounted on the wall of the measurement compartment. The information can in this manner be clearly reproduced in sufficient size so that a user can recognize the information from most points in the measurement chamber.

In a preferred embodiment the at least one item of information that is presented is adapted to an identity of the user. In this manner the information shown to a user can be matched to the identity of the user and adapted to the specific desires and requirements of this user. The presented information thus can be adapted to the identity of the user, in particular in a sequence of items of information. Given experienced and skilled users, for example, only the essential information critical for the operation can be shown, while all information necessary for the operation can be shown to users and beginners in need of instruction.

The at least one item of information can be modified by the user. In this manner the user can personally establish which information is essential for him or her for another user, and thus should be shown. A user can modify the shown information according to his or her wishes, in particular given a sequence of information. For example, a user can remove from the sequence of information the information that he or she deems to be non-essential for himself or herself (or also for another user) since the work step that is supported by this information is already well handled by the user. In contrast, the user can add to the sequence of information further information that he or she deems to be helpful for aiding the operation.

The at least one shown item of information is modified when an operator error has been determined by the medical imaging system. The shown information indicating the operator error can in this manner be adapted so that a user is instructed of errors committed previously given a later implementation of the method.

The at least one shown item of information is preferably stored in a memory of a control unit of the medical imaging system. The at least one shown item of information can be administered in a simple manner and can be loaded from the memory of the medical imaging system for the presentation. Storage of the sequence of information in the memory of the medical imaging system in particular proves to be advantageous when a user is shown a sequence of information adapted to the user. The sequence of information can be stored, for example, in a databank and thus be administered and adapted in a simple manner.

In an embodiment of the method, the operating state of the medical imaging system is determined using hardware of the medical imaging system. Hardware components (for example a switch or a sensor) can be arranged on specific components of the medical imaging system such that they are activated when a work step is effected at the components, for example when a patient bed is loaded with a patient or when an x-ray film has been inserted into a cassette. The internal operating state of the medical imaging system is changed in this manner according to the execution of specific work steps; the shown information is correspondingly adapted.

In a further embodiment of the method, the internal operating state of the medical imaging system is determined with the control software of the medical imaging system. In this manner the operating state can also be determined and the information correspondingly adapted without direct actuation of hardware components.

When the user is shown a sequence of items of information, the user can preferably initiate a change of one shown item of information for the next item of information of the sequence. Through the possibility to manually change the shown information, a user can switch along to the next item of information when he has already read and taken note of one item of information. The efficacy of the presentation of the information is increased in this manner.

The above object also is achieved by a medical imaging system operable according to the method described above, including all embodiments thereof.

### DESCRIPTION OF THE DRAWINGS

FIG. 1 schematically illustrates a magnetic resonance apparatus with which a user is directed in the operation of the magnetic resonance apparatus in accordance with the invention.

FIG. 2 is a flowchart of an embodiment for presenting a sequence of information for operation of a magnetic resonance apparatus to a user in accordance with the invention.

### DESCRIPTION OF THE PREFERRED EMBODIMENTS

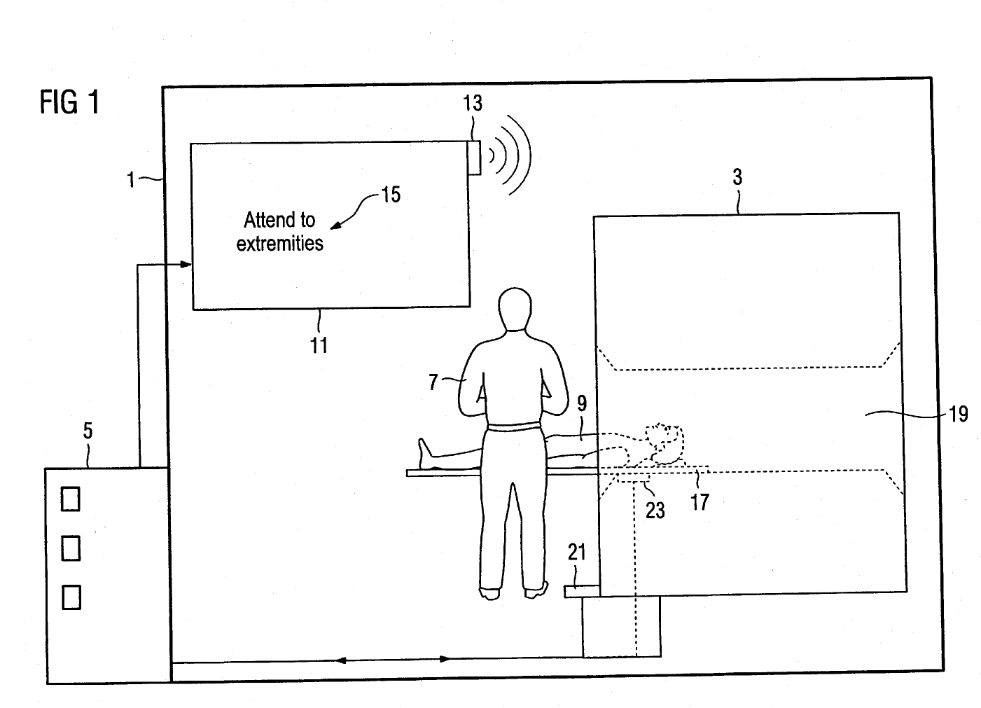

FIG. 1 illustrates a radio-frequency-shielded measurement compartment I in which the components of a magnetic resonance apparatus 3 are located with which the actual measurement is implemented. A control unit 5 for the magnetic resonance apparatus 3 is arranged outside of the measurement compartment 1.

For correct operation of the magnetic resonance apparatus 3 in the measurement compartment 1, a user must execute many steps and thus pay attention to the correct operation so that neither is the magnetic resonance apparatus 3 damaged nor is a patient 9 harmed.

So that the user 7 is directed in the correct implementation of the work steps, an imaging unit 11 in the viewing range of the user 7 as well as a sound output unit 13 are arranged in the measurement compartment 1. The imaging unit 11 can be, for example, a flat screen or a projection surface for a projector using which optical information can be output, which optical information supports the user 7 in the operation of the magnetic resonance apparatus 3. The sound output unit 13 is, for example, a speaker from which corresponding acoustic information is emitted.

The presented information 5 depends on an internal operating state of the magnetic resonance apparatus 3 such that the respective information that is significant for correct operation of the magnetic resonance apparatus 3 can be shown to the user 7. In the example shown in FIG. 1, the patient 9 (who has been positioned on a patient bed 17) is moved directly into the tunnel-like opening 19 of the magnetic resonance apparatus 3. At this point in time it is important for the user 7 to pay attention so that the patient 9 does not extend any extremities that can be crushed or jammed. So that the user 7 does not forget to attend to this detail, the corresponding information 15 is output on the imaging unit 11 and from the sound output unit 13 at the point in time at which the patient bed 17 with the patient 9 is moved into the opening 19.

Other items of information are output at other points in time. For example, after the patient 9 has been positioned on the patient bed 17 an instruction can appear that surface coils must be placed and that, if applicable, a pillow must be placed underneath in order to avoid burns due to radio-frequency energy or radio-frequency loops. After the surface coils have been placed, this is detected by the magnetic resonance apparatus 3 and other information is now superimposed, for example the instruction that an emergency ball must now be given to the patient and its mode of operation explained, or that headphones must be placed on the patient.

Which information is respectively shown depends on the internal operating state of the magnetic resonance apparatus 3 that can be varied and determined in different manners.

One possibility to determine the internal operating state of the magnetic resonance apparatus 3 is the use of hardware components. Such a hardware component can be, for example, a sensor that is arranged on the patient bed 17 and that detects the position and the movement of the patient bed 17 and transmits the detected data to the control unit 5 of the magnetic resonance apparatus 3. The control unit 5 thereupon adapts the shown information.

However, hardware components can also be used in order to present to the user 7 the possibility to influence the internal operating state and therewith also the shown information 15 itself. Shown here is, for example, a foot switch 21 that the user 7 can activate in order to switch from one item of information to the next item of information. In this manner, the user 7 can signal to the magnetic resonance apparatus 3 that he has already noted a specific item of information and would like to have presented the next item of information.

In addition to the use of hardware components, the internal operating state of the magnetic resonance apparatus 3 can also be determined by the control software of the control unit 5. For example, it is reasonable to superimpose specific information at the beginning or after the end of the actual measurement procedure. The end of the actual measurement procedure itself is determined by the control software without a hardware component being activated.

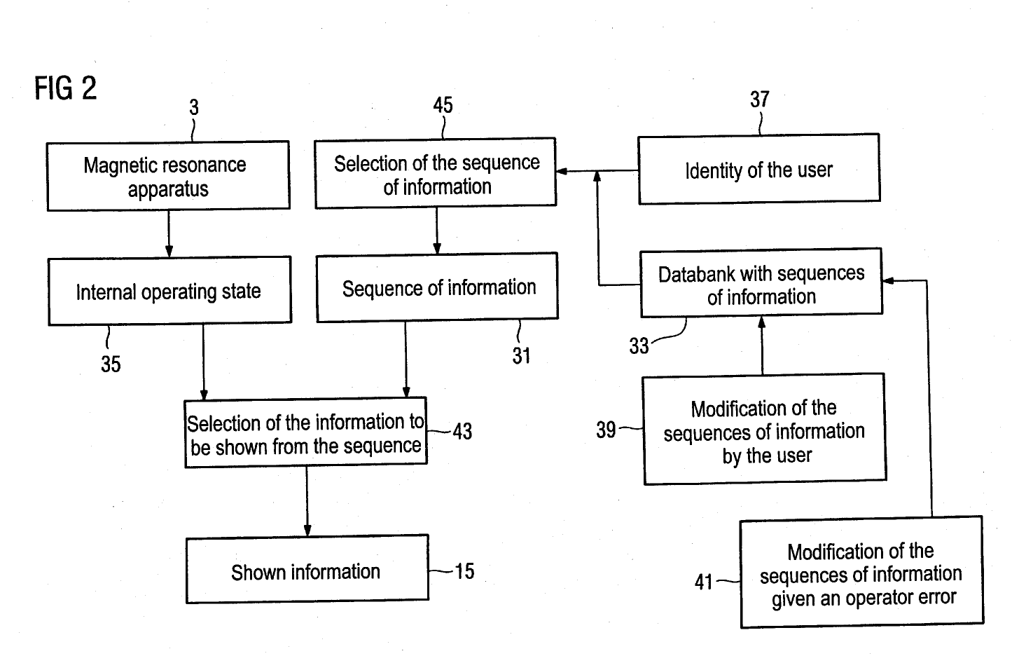

FIG. 2 schematically shows the method workflow of an embodiment of the method in which a sequence 31 of information is sequentially presented to the user 7. The shown information 15 is selected from the sequence of information 31 (selection of the information 43 to be shown), the selection ensuing dependent on the internal operating state 35 of the magnetic resonance apparatus 3. As further described using examples, the internal operating state 35 can be determined in various manners (aided by software and/or hardware).

The sequence 31 of information is for its part extracted from a databank 33 in which various sequences of information are stored. The various sequences of information stored in the databank 33 differ in different manners.

For example, the sequences of information differ in their degree of detail. Detailed sequences of information can thereby be helpful for unpracticed users while sequences of only essential information are better suited for experienced users 7 since such users are not distracted by superfluous information in this manner. Sequences of information in various languages can likewise be stored in the databank 33 so that users 7 with different linguistic backgrounds can be supported. The individual items of information can also be linked with graphical symbols (for example pictograms) so that a user 7 is also directed in a graphic manner using pictograms in addition to visually, textual instructions.

Which of the various sequences of information is selected from the databank 33 for the direction of a user 7 (selection of the sequence of information 45) is determined by the identity 37 of the user 7. After input of the identity 37 of the user 7, the sequence 31 of information desired by this user 7 is loaded from the databank 33.

The databank 33 additionally possesses means for administration of the databank 33 so that a user can modify a sequence of information stored in the databank 33 in a targeted manner according to his or her wishes (modification by user 39).

In a special embodiment, the magnetic resonance apparatus has a detector that detects an operator error. As soon as an operator error by a user has been detected, the sequence of information presented to the user is changed to the effect that the work step in which the operator error has occurred is supported by a corresponding instruction (modification given an operator error 41).

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for guiding a user through a data acquisition procedure involving a medical imaging system comprising the steps of:
under supervision by a user, operating a medical imaging system to interact with an examination subject to acquire data from the examination subject; and
during acquisition of said data with said medical imaging system, presenting at least one item of information, dependent on an internal operating state of the medical imaging system, to the user at the medical imaging system in a humanly perceptible form.

2. A method as claimed in claim 1 comprising presenting a sequence of items of information to the user regarding operation of the medical system, with the items of information in the sequence respectively depending on different internal operating states of the medical imaging system.

3. A method as claimed in claim 1 comprising presenting said at least one item of information in a humanly perceptible form selected from the group consisting of acoustical information and optical information.

4. A method as claimed in claim 1 wherein said medical imaging system is located in a measurement chamber, and presenting said at least one item of information to the user in said measurement chamber.

5. A method as claimed in claim 4 comprising presenting said at least one item of information on a wall of the measurement chamber.

6. A method as claimed in claim 1 comprising automatically electronically adapting said at least one item of information to an identity of the user.

7. A method as claimed in claim 1 comprising allowing the user to modify said at least one item of information to be presented to the user.

8. A method as claimed in claim 1 comprising detecting an operator error during the acquisition of said data by the user, and automatically modifying said at least one item of information dependent on said operator error.

9. A method as claimed in claim 1 comprising automatically electronically controlling presentation of said at least one item of information with a control unit having access to a memory, and storing said at least one item of information in said memory.

10. A method as claimed in claim 1 comprising determining said internal operating state of the medical imaging system using hardware of the medical imaging system.

11. A method as claimed in claim 1 comprising determining said internal operating state of the medical imaging system using control software of the medical imaging system.

12. A method as claimed in claim 1 comprising presenting a sequence of items of information to the user, said items of information in said sequence being respectively dependent on different internal operating states of the medical imaging system, and allowing a user to initiate a change from an item of information in said sequence to a next item of information in said sequence.

13. A medical imaging system comprising:
a data acquisition system operable to interact with an examination subject, under supervision of a user, to acquire medical imaging data from the examination subject; and
a control unit connected to the data acquisition system that monitors operation of the medical imaging system, and that presents at least one item of information to the user at the medical imaging system dependent on an internal operating state of the medical imaging system, during acquisition of said medical imaging data.
